(19) United States
(12) Patent Application Publication
(10) Pub. No.: US 2002/0188261 A1
(43) Pub. Date: Dec. 12, 2002

(54) Hand piece for use in a dermal abrasion system

(75) Inventor: Thomas Hruska, Laguna Woods, CA (US)

Correspondence Address: Richard R. Muccino, 758 Springfield Avenue, Summit, NJ, 07901, US

(73) Assignee: Dermacel Inc., San Clemente, CA

(21) Appl. No.: 09/876,920

(22) Filed: Jun. 7, 2001

(51) Int. Cl. A61M 35/00

(52) U.S. Cl. 604/289

(57) ABSTRACT

The present invention is directed to a substantially cylindrical hand piece for removing surface portions of skin by superficial abrasion caused by striking the skin with a mixture of air and a granular abrasive substance. Specifically, the invention pertains to a hand piece having a multi-port vacuum restrictor to create a vacuum vortex providing enhanced distribution of abrasive crystals. The present invention also includes an apparatus for use in a dermal abrasion system employing the novel hand piece.

### FIELD OF THE INVENTION

The present invention is directed to a substantially cylindrical hand piece for removing surface portions of skin by superficial abrasion caused by striking the skin with a mixture of air and a granular abrasive substance. Specifically, the invention pertains to a hand piece having a multi-port vacuum restrictor to create a vacuum vortex providing enhanced distribution of abrasive crystals. The present invention also includes an apparatus for use in a dermal abrasion system employing the novel hand piece.

### DESCRIPTION OF THE BACKGROUND

Dermal abrasion systems are used to remove a surface portion of skin to obtain aesthetic improvements in such are as as scars subsequent to a surgical procedure, burns, acne, scars received from photo sensitizing agents, small surface roughness on the face, and the like. Dermal abrasion systems apply a controlled application of a mixture of air and a granular abrasive substance, such as aluminum oxide, to the skin to obtain a superficial abrasion of adjustable magnitude. The dermal abrasion systems generally comprise a pressurized air supply, a vacuum supply, a supply canister for a granular abrasive substance, a hand piece to be applied to the surface of skin, and a waste collection canister for receiving the used abrasive substance and the removed portions of surface tissue from the skin. The abrasion may be of minimum value, such as a “peeling ” involving the removal of the outer-most layers of the epidermis, or may be of maximum value, such as a deep abrasion involving the dermis.

U.S. Pat. No. 3,750,909 (Butler) discloses an aerosol dispenser having a main valve and an auxiliary valve to clean out the nozzle coupled to the main valve. After use, nozzle 16 is removed from main valve 40 (FIG. 2) and coupled to auxiliary valve 56 (FIG. 3). By depressing the nozzle on the auxiliary valve, pressurized gas is forced through the nozzle cleaning it out. Specifically, the aerosol container comprises a closed container having a flowable material and pressurized gas for propelling the material out of the container. A main valve is mounted on the container above the level of the material and is connected to a tube which extends downward below the level of material. The main valve is normally open to allow the gas to propel the material through the tube and the valve out of the container. A nozzle is mounted on the main valve and coupled to direct the material out of the container. An auxiliary valve is provided in the container at a location at which the valve can be placed in direct communication with the gas. The nozzle can be cleaned out by the pressurized gas when the nozzle is coupled to the auxiliary valve and the valve is opened.

U.S. Pat. No. 4,358,027 (Poitras) discloses a liquid dispenser apparatus for dispensing metered quantities of a liquid. The apparatus comprises a container for the liquid to be dispensed and a pump having a cylinder with inlet and outlet ports which is manually reciprocal between an intake stroke, during which liquid is drawn through the inlet port, and a discharge stroke, during which the entire liquid content of the cylinder is expelled through the outlet port. The pump has a pump rod fixed to the piston and extends out of the cylinder to produce the intake and discharge strokes. A manual selector limiter is provided for adjusting the maximum length of the discharge stroke. The limiter has a slidable stop mounted on the pump rod so as to limit the discharge stroke and establish the volume of liquid drawn into the cylinder during the intake stroke. A fastener is supported by the stop and is movable between a locked position and a release position that allows the movement. An activator moves the fastener between the locked and release positions. An abutment surface on the cylinder engages the stop to limit the discharge stroke. The activator is operable to overcome the bias and force the fastener into the release position. The manual valve selectively opens and closes the inlet and outlet ports.

U.S. Pat. No. 5,050,782 (Cheng) discloses a measured volume liquid dispenser having an elongated barrel and plunger slidably mounted within a cylindrical bore in the barrel. The barrel has an inlet check valve located at the bottom of the cylindrical bore so that liquid may be drawn into the bore during an up-stroke of the plunger. The plunger has an axial passage and an outlet check valve in the passage. The outlet check valve allows liquid to be dispensed as the plunger is depressed in a downstroke in the barrel but provides a seal during the plunger upstroke so that the liquid may be drawn into the cylindrical bore through the inlet check valve.

U.S. Pat. No. 5,141,137 (Knodel) discloses a volumetric device for dispensing a predetermined volume of fluid from a fluid reservoir. The device includes a valve head mounted to a reservoir and a piston-valve pump mounted on the valve head. An intake valve and a discharge valve are each mounted to the valve head and are connected to the piston valve pump. Upon actuation of the piston valve pump, fluid is pumped from the reservoir through the intake valve and the valve head to the discharge valve. A ventilation valve has a first duct in the valve head connecting a blocking side of the ventilation valve to ambient atmosphere and a second duct connecting an opposite side of the ventilation valve with the reservoir. The ventilation valve is a check valve with a borehole in the direction of the vertical axis of the valve head. A free valve element is inserted into the borehole and is actuated by gravity toward sealing of a valve seat.

U.S. Pat. No. 5,254,108 (Burrell et al.) discloses a liquid applicator designed to be supported by hand for applying a liquid to a surface. The applicator has a handle which supports a roller equipped with a cam track and cam follower which reciprocates a piston within a cylinder to pump liquid to a nozzle metered with rotation of the roller.

U.S. Pat. No. 5,441,490 (Svedman) discloses an apparatus for use in the transdermal perfusion of fluids through the skin of a human. The apparatus has a housing attachable to the body with a contact surface held in contact with a portion of skin. The housing is a chamber and the contact surface is an aperture connected to the chamber. A fluid supply provides a perfusion phase and a de-epithelializing element provides for removal of a portion of epidermis. The de-epithelializing element exposes an area of dermis of the skin at a treatment site which is accessible via the aperture so that during the perfusion phase direct contact is made between the fluid in the chamber and the dermis.

U.S. Pat. No. 5,489,280 (Russell) discloses a fluid applicator configured to massage and irrigate a surgical site on a patient's skin. The applicator has a fluid reservoir for irrigating the surgical site and a collector for collecting irrigation fluids from the surgical site. A motor assembly is connected to the fluid reservoir for expelling a fluid in the reservoir and irrigating the surgical site. The fluid reservoir has a nozzle for directing the fluid from the fluid reservoir to the surgical site. The collector has a sponge connected to the fluid reservoir for engaging the surgical site when the applicator is used to massage and irrigate the surgical site. A means for returning fluids collected by the collector to the fluid reservoir is included.

U.S. Pat. No. 5,697,920 (Gibbons) discloses a device for cleaning the skin and its appendages. The device comprises a brush portion having soft bristles and two conduits. The first conduit has a connector distal to the brush for connection to a solution source and at the end proximal to the brush delivers the solution into the brush above the bristles. The second conduit has a negative pressure source and a first opening into the brush near the bristles of the brush.

U.S. Pat. No. 5,782,871 (Fujiwara et al.) discloses a sampling device of a suction effusion fluid which includes a cell having a vacuum suction port and a skin suction port. The vacuum suction port is connected to a vacuum source and the skin suction port is opposite to a skin surface. A slide valve in the cell is movable parallel to the skin surface and is used for opening or closing a path connecting the skin suction port with the vacuum suction port without releasing the vacuum source. Fluid reservoirs in the slide valve alternately connect with the skin suction port and the vacuum suction port through the slide valve. The fluid reservoirs store an effusion fluid from the skin surface by vacuum suction through the skin suction port. When the slide valve is moved to a position where a fluid reservoir is connected with the skin suction port and the vacuum suction port, one of the remaining fluid reservoirs is exposed to the outside of the cell.

U.S. Pat. No. 5,787,928 (Allen et al.) discloses a valve having a housing and a component movable within the housing. The housing has a first, second, third, fourth, and fifth ports and the movable component has a first passageway. A first orientation of the movable component within the housing connects the first port through the first passageway to the second port.

A second orientation of the movable component within the housing connects the second port through the first passageway to the third port. A second passageway is defined between the housing and the movable component. The fourth port and fifth port are connected to the second passageway at both the first and second orientations of the movable component. The fourth port comprises a first slot shaped opening facing the movable component.

U.S. Pat. No. 5,843,052 (Benja-Athon) discloses a compact irrigation and sterilization kit to cleanse and debride damaged structure such as a wound. The irrigation tubing kit has a flexible conduit having a coupling to attach the tubing kit to a faucet. A filter eliminates impurities from the flowing fluid from the faucet. A flow-regulating valve regulates the volume of the filter fluid flowing in the conduit. A short semi-rigid tubing extension with a short conduit and an opening is coupled to a vial of chemicals and medications to admit the chemicals and medications into the conduit. The flow of the chemicals and medications is regulated by another flow-regulating valve. Another short semi-rigid bypass port extension with a short conduit and an opening introduces fluids and chemical substances into the conduit of the tubing kit beyond the filter. A disperser distributes the chemical, medications and fluid over the wound. A reservoir which stores chemicals and medications prior to the application of the tubing kit delivers the chemicals and medications into the conduit of the tubing kit. The flowing fluid from a faucet flows through the coupling, filter and flow-regulating valve and provides the force to generate filtered fluid to remove foreign bodies from and to dispense the chemicals and medications from the reservoir directly into the conduit to sterilize the wound.

U.S. Pat. No. 5,848,998 (Marasco, Jr.) discloses a method for treating a body wound site on a patient which comprises applying a flexible transparent envelope about the wound site of the patient. The envelope has at least one primary opening with a peripheral lip to permit attachment of the envelope to the patient. A drain is connected to the envelope and a movable hand manipulatable fluid applying tissue treatment gun is introduced into the envelope. A fluid is sprayed into the envelope from the hand manipulable gun against the wound site for treatment of the patient. The envelope is spaced from the wound site by introducing a pressurized gas into the envelope. The pressurized gas is within the envelope during extended periods of fluid treatment and periods of absence of fluid treatment to prevent the envelope from contacting the wound site and to maintain the sterility of the wound site.

U.S. Pat. No. 5,862,958 (Edwards et al.) discloses a dispenser for connection to a mouth of a container for dispensing a liquid from the container. The dispenser comprises a dispensing fluid path connected to a dispensing orifice, a return fluid path connected to the liquid in the container, a cylinder having one end connected with the liquid in the container, and a plunger slidably mounted in the cylinder. Retracting the plunger in the cylinder moves the liquid from the container into the one end of the cylinder. A flow control valve has a path connecting one end of the cylinder and a control valve outlet and is movable between a first position placing the control valve outlet in connection with the dispensing path, whereby advancing the plunger in the cylinder moves the liquid from one end of the cylinder and out the dispensing orifice, and a second position placing the control valve outlet in connection with the return path, whereby advancing the plunger in the cylinder moves the liquid from the one end of the cylinder and back into the container. A first check valve in the control valve fluid path within the flow control valve permits a flow of the liquid from the one end of the cylinder to the control valve outlet and prevents the flow of liquid from the control valve outlet back into the one end of the cylinder. A second check valve is in the main passage and permits the liquid to flow from the container into the cylinder and prevents the liquid from flowing from the one end of the cylinder back into the container.

U.S. Pat. No. 5,037,432 (Molinari) discloses a hand tool or hand piece for removing skin by superficial abrasion with a mixture of air and a granular abrasive substance. The tool has an elongated manipulative body, a supply tube for the mixture, and a collection tube for the mixture and the removed portions of skin. Each tube has an operating head secured to the manipulative body. The head has a longitudinal axis and a wall with a portion inclined and offset with respect to the axis. A throughhole in the wall portion internally connects with the free ends of the terminal portions of the supply tube. The throughhole is aligned with the terminal portion of the supply tube so that by keeping in contact the throughhole with the surface, the stream of mixture travelling through the terminal portion of the supply tube and the throughhole is caused to strike the surface with an inclined incident angle.

U.S. Pat. No. 3,930,505 (Wallach) discloses an apparatus for removing animal tissue from a preselected area. The apparatus comprises a hand manipulatable first tube having a distally disposed outlet port, a liquid pulsating pump having an inlet and an outlet, a source of liquid connected to the pump inlet, a flexible conduit connecting the first tube to the pump outlet, a suction conduit including an outlet port disposed proximate the first tube outlet port, a source of suction, and a flexible conduit connecting the suction conduit to the source of suction. The apparatus may be used to remove defective, such as from the lens of an eye, by directing a pulsating high velocity liquid jet onto the defective tissue to disintegrate the tissue and sucking the liquid entraining the disintegrated tissue from the area adjacent the tissue by a suction conduit.

U.S. Pat. No. 4,715,848 (Beroza) discloses a gastric lavage device comprising a nozzle, an aspirating chamber in the nozzle, a window in a side of the aspirating chamber to communicate with a body cavity, an exit port in a rear portion of the nozzle, and an opening in a forward end of the aspirating chamber. The opening is directed toward the exit port from front to rear across the aspirating chamber. A source of pressurized liquid is connected to the opening and the opening and the source of pressurized liquid directs a stream of liquid through the aspirating chamber toward the exit port. The debris in the aspirating chamber is broken up and sent toward the exit port.

Reduced pressure is provided at the exit port so that debris in the body cavity is drawn through the window into the aspiration chamber to be broken up by the stream of liquid and removed through the exit port.

U.S. Pat. No. 5,029,580 (Radford et al.) discloses an aspirating apparatus for respiratory therapy in a human lung. The apparatus comprises a proximal end comprising a first site means further comprising means for selectively communicating a negative fluid pressure to the lung and second site means further comprising means for selectively communicating a positive pressure gas flow to and within the lung. A multi-purpose lung receiving aspirating catheter tube for respiratory therapy comprises secretion aspirating lumen means. The apparatus also comprises first fluid communication means by which the aspirating lumen mean are placed in fluid communication with the negative fluid pressure communicating means. The aspirating lumen means comprises distal end entry port means by which secretions and gas from the lungs of a patient enter the aspirating lumen means under force of negative pressure. The multi-purpose aspirating catheter tube for further comprises second lumen means. The apparatus also comprises second fluid communication means by which the second lumen means are placed in fluid communication with positive pressure gas. The second lumen means comprises distal end effluent port means by which the gas under force of the positive pressure is introduced to the lungs of the patient. The distal end effluent port means are closely juxtaposed the distal end entry port means whereby an intermixing flow within a common region adjacent to the distal tip of the catheter tube is accommodated.

U.S. Pat. No. 5,037,431 (Summers et al.) discloses a surgical apparatus for removing diseased tissue. The apparatus comprises liquid jet producing means operably movable by a surgeon over tissue to be removed, fluid pressurization means for pressurizing fluid and delivering the fluid under pressure to the liquid jet producing means, the liquid jet producing means for operably producing a narrow jet of pressurized fluid to be directed toward the tissue to be removed by the surgeon, the jet producing means being adapted for removing tumors from skin wherein the tumors have fingers that extend under healthy skin tissue, and including angled nozzle means secured to a liquid jet exiting end of the jet producing means directing the jet of pressurized fluid at an angle other than perpendicular to skin tissue so as to allow for removal of the tumor fingers by the jet, and rotating means positioned within the apparatus for cooperating with and rotating the nozzle means such that a point of impact of the jet describes a generally circular pattern relative to the tissue during use.

U.S. Pat. No. 5,123,902 (Müller et al.) discloses an apparatus for performing surgery on biological pathological tissue in a region of nonpathological tissue with the aid of a laser beam applied to the pathological tissue. The apparatus comprises a laser for generating the laser beam with a predetermined wavelength. A transmission means is provided for transmitting the laser beam to the site of the operation to produce a laser focus wherein the energy density in the focus is above a level to cause photoablation whereby secondary radiation is produced capable of causing damage to the nonpathological tissue. The secondary radiation is radiation of the laser beam backscattered from the eye or fluorescence radiation of the tissue. A suction/irrigation means removes the tissue ablated by the laser beam. A metering unit meters an organic-tissue penetrating substance to the site for absorbing the secondary radiation and to provide a reduced effective threshold for removing by photoablation the pathological tissue. The substance is absorbent in the range of the wavelength of the laser beam and which increases photoablation while at the same time protects the nonpathological tissue not treated by the laser beam against damage by the secondary radiation.

Measuring means for measuring the secondary radiation backscattered from the tissue detects when the backscattered radiation drops or exceeds predetermined limit values selected to reduce damage to the nonpathological tissue.

U.S. Pat. No. 5,207,234 (Rosso) discloses a method for making microabrasions on human tissue. The method comprises placing a handle having an open chamber on human tissue to close the chamber, applying suction to the chamber to sealingly engage the periphery of the chamber with the human tissue and to apply a suction force to the human tissue toward the chamber, inducing the flow of air into the chamber only by the removal of air from the chamber by suction while restricting entry of ambient air from the surrounding atmosphere into the chamber around the periphery of the chamber, entraining a reducing substance in the flow of air prior to entry of the flow of air into the chamber, directing substantially all the reducing substance entering the chamber directly against the human tissue at an angle of substantially 45°, entraining used reducing substances and abraded tissue in the flow of air being removed from the chamber by suction, and collecting used reducing substances and abraded tissue in a manner to prevent reuse of the reducing substances during continued application of a vacuum to the chamber.

U.S. Pat. No. 5,318,518 (Plechinger et al.) discloses an irrigating catheter for eliminating solids from body organs and hollow body cavities. The irrigating catheter comprises a catheter body having a first lumen and a second lumen, the first lumen being a transporting lumen for supplying an irrigating fluid from a high-pressure fluid source to an organ, the second lumen being a discharging lumen for discharging the irrigating fluid and the solids from the organ that have been entrained by the irrigating fluid. A nozzle is formed at an outlet of the transporting lumen and is disposed at a distance from and opposite to an inlet of the discharging lumen. The nozzle sends into the inlet an irrigating fluid jet which engages the solids located between the nozzle and the inlet and discharges the solids through the discharging lumen. The discharging lumen has a mixing tube, an inlet of which is the inlet of the discharging lumen and in which the solid particles can disperse in the irrigating liquid, a diffuser, and a discharging duet. The mixing tube and the diffuser are dimensioned such that the diffuser brings about an intensification of the suction effect of the irrigating fluid jet at the inlet of the mixing tube.

U.S. Pat. No. 5,320,599 (Griep et al.) discloses a drainage catheter which comprises a flexible tubular basic catheter body defining separate catheter lumens respectively. The catheter comprises a pressure channel and a discharge channel, connector means at a proximal end of the catheter for connecting the pressure channel to a source of liquid under pressure and the discharge channel to discharge means. An inlet opening is positioned in the side of the catheter adjacent the distal end. A discharge channel communicates with the inlet opening. The pressure channel extends from the proximal end distally forward of the inlet opening and the curving rearwardly to join the discharge channel at the inlet opening. The pressure channel defines a spray nozzle to direct pressurized fluid in the pressure channel across the inlet opening and into the discharge channel to create a suction adjacent the opening. The spray nozzle is narrowed at one end to form a jet, the one end being positioned to point across the inlet opening and into the discharge channel. A narrow passage opening is defined on a side remote from the inlet opening and adjacent the distal end in an outer will portion of the catheter between the pressure channel and the exterior.

U.S. Pat. No. 5,453,088 (Boudewijn et al.) discloses a drainage catheter comprising a basic tubular body having a distal end, a proximal end, and a pressure channel in the tubular body and a discharge channel in the tubular body. The distal end has a bending of the pressure channel to communicate with the discharge channel. A nozzle ejector formation in the distal end of the catheter after the bend in the pressure channel has an orifice facing proximally into the discharge channel. The distal end further includes an inlet opening disposed adjacent the orifice of the nozzle ejector formation. The basic tubular body has, at the proximal end, a pressure inlet to the pressure channel and a discharge outlet from the discharge channel. A coupling means is coupled to the proximal end of the basic tubular body and includes a first, pressure channel for coupling a source of fluid under pressure to the pressure inlet at the proximal end of the catheter tubular body, A second, discharge channel couples the discharge outlet at the proximal end of the tubular body to a reservoir. A third, curved channel couples, at the pressure inlet, the source of fluid under pressure to, at the discharge outlet, the reservoir. Fluid under pressure flows through the third channel creating a suction at the discharge outlet to reduce fluid from the discharge channel to the reservoir.

U.S. Pat. No. 5,527,330 (Tovey) discloses a surgical instrument for cutting tissue and suctioning. The instrument is adapted for coupling to an external high pressure fluid source and an external vacuum source. The instrument comprises an elongated shaft defining a longitudinal axis and having a proximal end portion and a distal end portion. An irrigation tube is disposed within the elongated shaft and has a proximal end portion and a distal end portion, the proximal end portion configured for coupling to an external high pressure fluid source, the distal end portion disposed beyond the elongated shaft and defining a nozzle portion to direct a stream of high pressure fluid supplied by the high pressure fluid source to cut tissue. A suction tube is disposed within the elongated shaft and has a proximal end portion and a distal end portion, the proximal end portion configured for coupling to an external vacuum source, the distal end portion disposed beyond the elongated shaft and positioned and oriented in an opposed spaced relation relative to the nozzle portion of the irrigation tube such that body tissue is positionable therebetween. The distal end portion has a vacuum opening configured to receive and suction away waste fluid and separated tissue.

U.S. Pat. No. 5,788,667 (Stoller) discloses a microsurgical vitreacmy fluid jet cutting device for cutting and removing vitreous from an eye. The device comprises a substantially rigid casing tube defining a chamber. The casing tube has a first vacuum port communicating with the chamber proximate a first end of the tube and an opening through the tube proximate a second end. A vacuum means applies a vacuum to the chamber through the first vacuum port to draw the vitreous into the chamber through the opening. A fluid jet supply tube extends in the casing tube and has a discharge located within the chamber adjacent the opening oriented to discharge a fluid jet across a path through which the vitreous is drawn. A fluid jet cuts the vitreous drawn through the opening and the cut vitreous is drawn from the chamber through the vacuum port. A hand grip is connected to the casing tube for concurrently manipulating the hand grip and the casing tube.

U.S. Pat. No. 5,810,842 (DiFiore et al.) discloses a device for microdermoabrasion using a flow of a mixture of air and a reducing substance. The device comprises a casing, a vacuum pump at an interior of the casing, a compressor at an interior of the casing, a control footswitch operatively connected to the compressor for actuating the compressor, a mixing bulb at an exterior of the casing, a collecting bulb at an exterior of the casing, and a handpiece extending between the mixing bulb and the collecting bulb. The mixing bulb contains a mixture of air and reducing substances. The mixing bulb, the handpiece, and the collecting bulb each comprise a single monoblock and together form a single unit which is detachably connected to the casing.

U.S. Pat. No. 5,971,999 (Naldoni) discloses an apparatus for microdermoabrasion by means of a jet of a mixture of air and crystals. The apparatus comprises a main body having a vacuum pump, a mixing container wherein the air is mixed with the crystals, the mixing container including a first disposable cartridge full of unused crystals and a first locking means having a mixing cannula and a throttle valve. A recovering container is provided wherein the crystals are recovered after use and includes a second disposable cartridge which is identical to the first disposable cartridge. A second locking means has a disposable first filter and a duct. A handle for applying the mixture of air and crystals is also provided. The first disposable cartridge, once used and emptied, is utilized as the second disposable cartridge.

U.S. Pat. No. 5,980,512 (Silberg) discloses a system for resurfacing skin. The system comprises a laser adapted to emit a laser beam against a skin site of a patient, and a bathing means for immersing the skin site in an oxygen replacing gas. The bathing means includes a containment mechanism securable to the skin of the patient and adapted to encircle the skin site. The containment mechanism encapsulates the skin site and has a laser permeable window.

### IN THE FIGURES

FIG. 1 illustrates a side view of a hand piece for use in a dermal abrasion system in a preferred embodiment of the present invention.

FIG. 2 illustrates a schematic view of multiport flow restrictors for use in a dermal abrasion system in a preferred embodiment of the present invention. FIG. 2A illustrates a first multiport flow restrictor in the second portion of the supply tube to restrict flow of the mixture and thereby provide enhanced distribution of the granular abrasive substance. FIG. 2B illustrates a second multiport flow restrictor in the second portion of the collection tube to restrict flow of the mixture and thereby provide enhanced distribution of the granular abrasive substance.

### SUMMARY OF THE INVENTION

The present invention is directed to a substantially cylindrical hand piece for removing surface portions of skin by superficial abrasion caused by striking the skin with a mixture of air and a granular abrasive substance, wherein the hand piece comprises:

(a) a supply tube for supplying the mixture; and

(b) a collection tube for collecting the mixture and removed portions of skin;

wherein the supply tube and the collection tube each have a first portion and a second portion; the first portion of the supply tube connects to a supply canister supplying the mixture of air and granular abrasive substance and the first portion of the collection tube connects to a collection canister collecting the mixture and removed portions of skin; the second portion of the supply tube and the second portion of the collection tube communicate with each other; a throughhole in the second portion of the supply tube is aligned with the surface portion of skin to be removed such that the mixture travelling through the throughhole strikes the surface portion of skin at an inclined incident angle; and a first multiport flow restrictor in the second portion of the supply tube restricts flow of the mixture and thereby provides enhanced distribution of the granular abrasive substance.

In another embodiment, the invention is directed to an apparatus for use in a dermal abrasion system comprising:

(A) a pressurized air supply;

(B) a vacuum supply;

(C) a supply canister for a granular abrasive substance employing a purge button to remove debris clogging the system;

(D) a waste canister;

(E) a substantially cylindrical hand piece for removing surface portions of skin by superficial abrasion caused by striking the skin with a mixture of air and a granular abrasive substance; and

(F) duct means for conveying pressurized air from the pressurized air supply to the supply canister, duct means for conveying a mixture of air and granular abrasive substance from the supply canister to the hand piece, duct means for conveying the mixture of air and granular abrasive substance and removed portions of skin from the hand piece to the waste canister, and duct means from the waste canister to the vacuum supply;

wherein the supply canister comprises:

(a) a housing having a top and a bottom;

(b) a feed inlet fitting on the top of the outside of the housing for introducing a granular abrasive substance into the housing;

(c) an air pressure inlet fitting at the bottom of the outside of the housing for introducing air pressure into the housing;

(d) a vacuum inlet fitting at the bottom of the outside of the housing for introducing vacuum into the housing;

(e) an air disperser inside the housing, wherein the bottom of the air disperser communicates with the air pressure inlet fitting and the top of the air disperser is configured to disperse air inside the housing; and

(f) a velocity feed tube inside the housing being open at the top and having a metering hole configured to receive a granular abrasive substance and positioned on the side of the velocity feed tube facing the air disperser, wherein the bottom of the velocity feed tube communicates with the vacuum inlet fitting and is configured to introduce vacuum inside the housing through the open top of the velocity feed tube and the metering hole, the open top of the velocity feed tube being above the level of granular abrasive substance, when present;

wherein the hand piece comprises:

(a) a supply tube for supplying the mixture; and

(b) a collection tube for collecting the mixture and removed portions of skin;

wherein the supply tube and the collection tube each have a first portion and a second portion; the first portion of the supply tube connects to a supply canister supplying the mixture of air and granular abrasive substance and the first portion of the collection tube connects to a collection canister collecting the mixture and removed portions of skin; the second portion of the supply tube and the second portion of the collection tube communicate with each other; a throughhole in the second portion of the supply tube is aligned with the surface portion of skin to be removed such that the mixture travelling through the throughhole strikes the surface portion of skin at an inclined incident angle; and a first multiport flow restrictor in the second portion of the supply tube restricts flow of the mixture and thereby provides enhanced distribution of the granular abrasive substance.

### DETAILED DESCRIPTION OF THE INVENTION

The present invention pertains to a dermal abrasion apparatus, or a particle skin resurfacing system, for the controlled removal of surface portions of human skin. The apparatus applies a controlled application of a reducing substance, such as aluminum oxide, to the skin to obtain a superficial abrasion of adjustable magnitude. The dermal abrasion apparatus comprises a pressurized fluid generator, a vacuum generator, a reducing substance supply canister with an intake hole, a hand piece to apply the reducing substance to the skin, and a collection canister for receiving the used reducing substance and the removed portions of surface tissue from the skin. A problem with conventional dermal abrasion apparatuses is that the hand piece in the reducing substance supply canister has just one port and thereby provides a “hot” spot when applying the reducing substance to the skin.

The present invention pertains to a hand piece to apply the reducing substance to the skin with enhanced distribution of abrasive crystals thereby eliminating the “hot” spot. A first multiport flow restrictor in the supply tube acts to create a vacuum vortex providing precise control and enhanced distribution of abrasive crystals with uniform skin abrasion thereby eliminating the “hot” spot of having just one port. If one port in the first multiport flow restrictor should become clogged, the increased vacuum on the other ports acts to free, or clear the clog. The convergent/divergent airstream maximizes the vacuum vortex effect. In a preferred embodiment, a second multiport flow restrictor is also provided in the collection tube to further create a vacuum vortex providing enhanced distribution of abrasive crystals with uniform skin abrasion. The multiport flow restrictors are preferably made of wear resistant ceramic and the hand piece is preferably made from tempered Pyrex glass. The improved hand piece with a multiport flow restrictor provides uniform abrasion with no spots, improved fluidics, and more effective tissue removal.

The present invention also includes an apparatus for use in a dermal abrasion system employing the novel hand piece button. The apparatus comprises a pressurized air supply, a vacuum supply, a supply canister for a granular abrasive substance, a waste canister, and a hand piece to be applied to the surface of the skin. The pressurized air supply is conveniently compressed air from a compressor which maintains air under pressure contained within a reservoir, a pressure regulator connected in series with an output from the reservoir, and a solenoid valve which allows the air under pressure to flow out along a duct. The vacuum supply is conveniently a vacuum pump which maintains a vacuum within a reservoir. The granular abrasive substance may be, for example, microcrystals of quartz, metal, dust or derivatives of aluminium such as aluminum oxide (corundum), possibly having different grain size diameters. The hand piece is an instrument manipulated by the medical operator to remove the portions of tissue. The hand piece has a supply tube with an inlet connected to the pressurized air supply and the supply canister, a throughhole in a head disposed along the axis of the supply tube in a position facing an outlet from the tube itself to permit the granular abrasive substance to strike the tissue removing the surface portions, and a collection tube connected to the vacuum supply for the purpose of removing both the used granular abrasive substance and the removed portions of surface tissue from the surface of the tissue.

The invention will be better understood from the following detailed description of the preferred embodiments taken in conjunction with the Figures, in which like elements are represented by like referenced numerals.

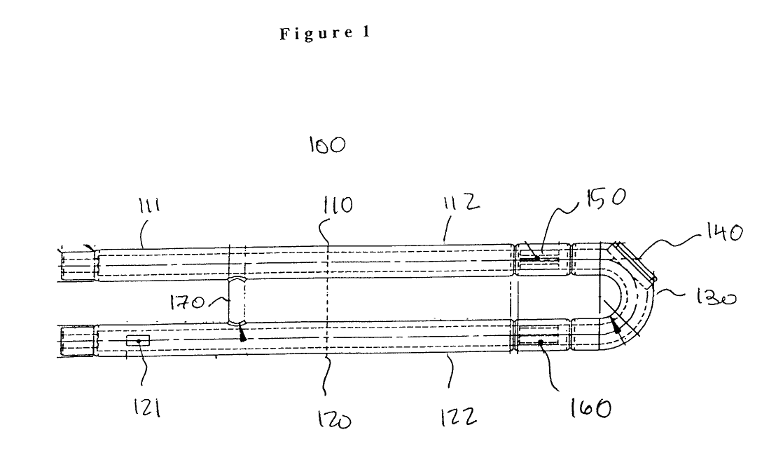

FIG. 1 illustrates a side view of a hand piece for use in a dermal abrasion system in a preferred embodiment of the present invention.

In FIG. 1, the hand piece is depicted generally as 100 and is constructed in accordance with a preferred embodiment of the present invention. Hand piece 100 is substantially cylindrical and includes a supply tube 110 for supplying the mixture and a collection tube 120 for collecting the mixture and removed portions of skin. The supply tube 110 has a first portion 111 and a second portion 112. The collection tube 120 has a first portion 121 and a second portion 122. The first portion of the supply tube 111 connects to a supply canister supplying the mixture of air and granular abrasive substance (not shown). The first portion of the collection tube 121 connects to a collection canister collecting the mixture and removed portions of skin (not shown). The second portion of the supply tube 112 and the second portion of the collection tube 122 communicate with each other at 130. A throughhole 140 in the second portion of the supply tube 112 is aligned with the surface portion of skin to be removed such that the mixture travelling through the throughhole 140 strikes the surface portion of skin at an inclined incident angle. A first multiport flow restrictor 150 in the second portion of the supply tube 112 restricts flow of the mixture and thereby provides enhanced distribution of the granular abrasive substance. Preferably, the first multiport flow restrictor 150 in the second portion of the supply tube has from 4 to 9 ports, more preferably, the first multiport flow restrictor 150 has 4 ports.

Optionally, the hand piece 100 may further comprise a second multiport flow restrictor 160 in the second portion of the collection tube 122 to restrict flow of the mixture and thereby provide enhanced distribution of the granular abrasive substance. Preferably, the second multiport flow restrictor 160 in the second portion of the collection tube has from 2 to 7 ports, more preferably, the second multiport flow restrictor 160 has 2 ports.

Optionally, the hand piece 100 may further comprise a coupling support 170 connecting and supporting the supply tube 110 and the collection tube 120. Preferably, the coupling support 170 connects and supports the first portion of the supply tube 111 and the first portion of the collection tube 121.

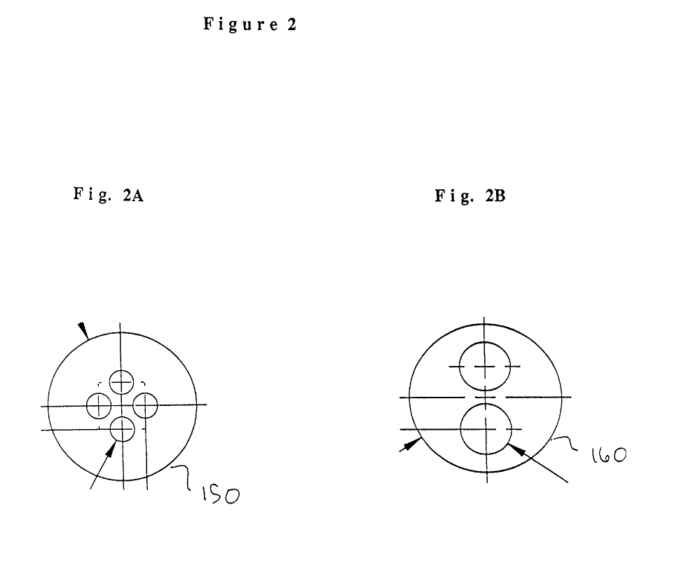

FIG. 2 illustrates a schematic view of multiport flow restrictors for use in a dermal abrasion system in a preferred embodiment of the present invention. FIG. 2A illustrates a first multiport flow restrictor for use in the second portion of the supply tube to restrict flow of the mixture and thereby provide enhanced distribution of the granular abrasive substance. FIG. 2A illustrates a preferred first multiport flow restrictor for use in the second portion of the supply tube having 4 ports. FIG. 2B illustrates a second multiport flow restrictor in the second portion of the collection tube to restrict flow of the mixture and thereby provide enhanced distribution of the granular abrasive substance. FIG. 2B illustrates a preferred second multiport flow restrictor for use in the second portion of the collection tube having 2 ports.

In a preferred embodiment, the invention pertains to an apparatus for use in a dermal abrasion system comprising:

(A) a pressurized air supply;

(B) a vacuum supply;

(C) a supply canister for a granular abrasive substance employing a purge button to remove debris clogging the system;

(D) a waste canister;

(E) a substantially cylindrical hand piece for removing surface portions of skin by superficial abrasion caused by striking the skin with a mixture of air and a granular abrasive substance; and

(F) duct means for conveying pressurized air from the pressurized air supply to the supply canister, duct means for conveying a mixture of air and granular abrasive substance from the supply canister to the hand piece, duct means for conveying the mixture of air and granular abrasive substance and removed portions of skin from the hand piece to the waste canister, and duct means from the waste canister to the vacuum supply;

wherein the supply canister comprises:

(a) a housing having a top and a bottom;

(b) a feed inlet fitting on the top of the outside of the housing for introducing a granular abrasive substance into the housing;

(c) an air pressure inlet fitting at the bottom of the outside of the housing for introducing air pressure into the housing;

(d) a vacuum inlet fitting at the bottom of the outside of the housing for introducing vacuum into the housing;

(e) an air disperser inside the housing, wherein the bottom of the air disperser communicates with the air pressure inlet fitting and the top of the air disperser is configured to disperse air inside the housing; and

(f) a velocity feed tube inside the housing being open at the top and having a metering hole configured to receive a granular abrasive substance and positioned on the side of the velocity feed tube facing the air disperser, wherein the bottom of the velocity feed tube communicates with the vacuum inlet fitting and is configured to introduce vacuum inside the housing through the open top of the velocity feed tube and the metering hole, the open top of the velocity feed tube being above the level of granular abrasive substance, when present;

wherein the hand piece comprises:

(a) a supply tube for supplying the mixture; and

(b) a collection tube for collecting the mixture and removed portions of skin;

wherein the supply tube and the collection tube each have a first portion and a second portion; the first portion of the supply tube connects to a supply canister supplying the mixture of air and granular abrasive substance and the first portion of the collection tube connects to a collection canister collecting the mixture and removed portions of skin; the second portion of the supply tube and the second portion of the collection tube communicate with each other; a throughhole in the second portion of the supply tube is aligned with the surface portion of skin to be removed such that the mixture travelling through the throughhole strikes the surface portion of skin at an inclined incident angle; and a first multiport flow restrictor in the second portion of the supply tube restricts flow of the mixture and thereby provides enhanced distribution of the granular abrasive substance.

The invention being thus described, it will be obvious that the same may be varied in many ways. Such variations are not to be regarded as a departure from the spirit and scope of the invention and all such modifications are intended to be included within the scope of the following claims.

We claim:

1. A substantially cylindrical hand piece for removing surface portions of skin by superficial abrasion caused by striking the skin with a mixture of air and a granular abrasive substance, wherein the hand piece comprises:
(a) a supply tube for supplying the mixture; and
(b) a collection tube for collecting the mixture and removed portions of skin;
wherein the supply tube and the collection tube each have a first portion and a second portion; the first portion of the supply tube connects to a supply canister supplying the mixture of air and granular abrasive substance and the first portion of the collection tube connects to a collection canister collecting the mixture and removed portions of skin; the second portion of the supply tube and the second portion of the collection tube communicate with each other; a throughhole in the second portion of the supply tube is aligned with the surface portion of skin to be removed such that the mixture travelling through the throughhole strikes the surface portion of skin at an inclined incident angle; and a first multiport flow restrictor in the second portion of the supply tube restricts flow of the mixture and thereby provides enhanced distribution of the granular abrasive substance.

2. The hand piece according to claim 1, wherein the first multiport flow restrictor in the second portion of the supply tube has from 4 to 9 ports.

3. The hand piece according to claim 2, wherein the first multiport flow restrictor in the second portion of the supply tube has 4 ports.

4. The hand piece according to claim 1, further comprising a second multiport flow restrictor in the second portion of the collection tube to restrict flow of the mixture and thereby provide enhanced distribution of the granular abrasive substance.

5. The hand piece according to claim 4, wherein the second multiport flow restrictor in the second portion of the collection tube has from 2 to 7 ports.

6. The hand piece according to claim 5, wherein the second multiport flow restrictor in the second portion of the collection tube has 2 ports.

7. The hand piece according to claim 1, further comprising a coupling support connecting and supporting the supply tube and the collection tube.

8. An apparatus for use in a dermal abrasion system comprising:
(A) a pressurized air supply;
(B) a vacuum supply;
(C) a supply canister for a granular abrasive substance employing a purge button to remove debris clogging the system;
(D) a waste canister;
(E) a substantially cylindrical hand piece for removing surface portions of skin by superficial abrasion caused by striking the skin with a mixture of air and a granular abrasive substance; and
(F) duct means for conveying pressurized air from the pressurized air supply to the supply canister, duct means for conveying a mixture of air and granular abrasive substance from the supply canister to the hand piece, duct means for conveying the mixture of air and granular abrasive substance and removed portions of skin from the hand piece to the waste canister, and duct means from the waste canister to the vacuum supply;
wherein the supply canister comprises:
(a) a housing having a top and a bottom;
(b) a feed inlet fitting on the top of the outside of the housing for introducing a granular abrasive substance into the housing;
(c) an air pressure inlet fitting at the bottom of the outside of the housing for introducing air pressure into the housing;
(d) a vacuum inlet fitting at the bottom of the outside of the housing for introducing vacuum into the housing;
(e) an air disperser inside the housing, wherein the bottom of the air disperser communicates with the air pressure inlet fitting and the top of the air disperser is configured to disperse air inside the housing; and
(f) a velocity feed tube inside the housing being open at the top and having a metering hole configured to receive a granular abrasive substance and positioned on the side of the velocity feed tube facing the air disperser, wherein the bottom of the velocity feed tube communicates with the vacuum inlet fitting and is configured to introduce vacuum inside the housing through the open top of the velocity feed tube and the metering hole, the open top of the velocity feed tube being above the level of granular abrasive substance, when present;
wherein the hand piece comprises:
(a) a supply tube for supplying the mixture; and
(b) a collection tube for collecting the mixture and removed portions of skin;
wherein the supply tube and the collection tube each have a first portion and a second portion; the first portion of the supply tube connects to a supply canister supplying the mixture of air and granular abrasive substance and the first portion of the collection tube connects to a collection canister collecting the mixture and removed portions of skin; the second portion of the supply tube and the second portion of the collection tube communicate with each other; a throughhole in the second portion of the supply tube is aligned with the surface portion of skin to be removed such that the mixture travelling through the throughhole strikes the surface portion of skin at an inclined incident angle; and a first multiport flow restrictor in the second portion of the supply tube restricts flow of the mixture and thereby provides enhanced distribution of the granular abrasive substance.

9. The apparatus according to claim 8, wherein the first multiport flow restrictor in the second portion of the supply tube has from 4 to 9 ports.

10. The apparatus according to claim 9, wherein the first multiport flow restrictor in the second portion of the supply tube has 4 ports.

11. The apparatus according to claim 8, further comprising a second multiport flow restrictor in the second portion of the collection tube to restrict flow of the mixture and thereby provide enhanced distribution of the granular abrasive substance.

12. The apparatus according to claim 11, wherein the second multiport flow restrictor in the second portion of the collection tube has from 2 to 7 ports.

13. The apparatus according to claim 12, wherein the second multiport flow restrictor in the second portion of the collection tube has 2 ports.

14. The apparatus according to claim 8, further comprising a coupling support connecting and supporting the supply tube and the collection tube.

15. The apparatus according to claim 8, further comprising a granular abrasive substance.
